(19) United States
(12) Patent Application Publication
(10) Pub. No.: US 2008/0092638 A1
(43) Pub. Date: Apr. 24, 2008

(54) Wireless analyte monitoring system

(75) Inventors: Allen J. Brenneman, Goshen, IN (US); Mihailo V. Rebec, Bristol, IN (US); James E. Smous, Niles, MI (US)

Correspondence Address: NIXON PEABODY LLP, 161 N. CLARK STREET, 48TH FLOOR, CHICAGO, IL, 60601, US

(73) Assignee: Bayer HealthCare LLC

(21) Appl. No.: 11/906,354

(22) Filed: Oct. 2, 2007

Related U.S. Application Data
(60) Provisional application No. 60/852,928, filed on Oct. 19, 2006.

(51) Int. Cl. G01N 33/49

(52) U.S. Cl. 73/61.41

(57) ABSTRACT

A system for monitoring a concentration of an analyte in a fluid or tissue sample. The system comprises a sensor module adapted to be borne on a patient. The sensor module includes a power supply adapted to provide a transmission power, a first transceiver adapted to transmit analyte-concentration information, and a memory. The system further comprises a remote monitoring device adapted to wirelessly communicate with the sensor module. The remote monitoring device includes a second transceiver adapted to receive the analyte-concentration information transmitted by the sensor module and adapted to transmit a signal to the sensor module confirming that the analyte-concentration information was received. The information is stored in the memory until the signal is received by the sensor module.

### CROSS-REFERENCE TO RELATED APPLICATIONS

This application claims priority to U.S. Provisional Application Ser. No. 60/852,928 filed on Oct. 19, 2006, which is incorporated by reference in its entirety.

### FIELD OF THE INVENTION

The present invention relates generally to a system and method for wirelessly monitoring the concentration of one or more analytes in a fluid or tissue sample.

### BACKGROUND OF THE INVENTION

The quantitative determination of analytes in body fluids is of great importance in the diagnoses and maintenance of certain physiological abnormalities. In particular, determining glucose in body fluids is important to diabetic individuals who must frequently check the glucose level in their body fluids to regulate the glucose intake in their diets.

In one current type of blood glucose testing system, test sensors are used to test a sample of blood. The testing end of the test sensor is placed into the blood that has, for example, accumulated on a patient's finger after the finger has been pricked. Blood samples are often taken from a fingertip of a test subject because of the high concentration of capillaries, which can provide an effective blood supply. The blood may be drawn into a capillary channel that extends in the test sensor from the testing end to the reagent material by capillary action so that a sufficient amount of blood is drawn into the test sensor. A voltage is applied, causing the glucose in the blood to then chemically react with the reagent material in the test sensor, resulting in an electrical signal indicative of the glucose level in the blood. This signal is supplied to a sensor-dispensing instrument, or meter, via contact areas located near the rear or contact end of the test sensor and becomes the measured output.

Drawing blood each time a glucose reading is desired is an inconvenient and invasive procedure. Moreover, drawing blood is undesirable because of the resulting pain, discomfort, and risk of infection often experienced by the patient each time a blood sample is taken.

To assist in minimizing the disadvantages associated with invasive analyte-testing procedures, the concentration of a desired analyte may be continuously monitored. Continuous monitoring generally includes a portable test sensor module being borne by a patient. The sensor module collects data, which may include a parameter correlated with the concentration of the measured analyte. The sensor module may be placed over the patient's skin. Alternatively, a portion (e.g., the testing portion) of the sensor module may be placed under the patient's skin. Although placing the sensor module underneath the patient's skin does not eliminate pain, discomfort, and/or risk of infection associated therewith, it may reduce these disadvantages because the patient need only be pricked once to obtain multiple measurement data or readings.

For added convenience, continuous analyte monitoring systems may be wireless. A portable sensor module of a wireless continuous monitoring system includes a transmitter to wirelessly transmit data signals to a remote monitoring device (RMD). The RMD may, for example, be worn around the neck of a patient using a neck strap, shoulder strap, belt clip, or other suitable means. The RMD generally includes a wireless receiver to receive the data signals from the sensor module, a mechanism to interpret the data collected by the sensor module, and a data memory for storage of the interpreted data. The RMD may be equipped to communicate with other devices including, but not limited to, devices that may modify the rate of intravenous drip of drugs, glucose, insulin, drugs, pain killers, chemotherapeutic agents, or the like based on information received from the RMD.

One problem associated with typical wireless continuous monitoring systems includes loss of data signals due to, for example, the receiver being out of range, being in an “off” position, interference in the communication link, or the like. Because the patient may become separated from the RMD, the data signal transmitted by the sensor module may not reach the RMD. Thus, analyte concentration measurements may be permanently lost. Thus, each sensor module must be maintained no more than a certain distance away from the RMD to ensure that each data measurement will be successfully transmitted.

Furthermore, wireless continuous monitoring systems typically include a single sensor module associated with a single RMD. Thus, the patient is generally the only person who receives immediate notification if dangerous analyte concentration levels are reached. This may be undesirable for several reasons. For example, an RMD is generally kept on or near a child whose glucose levels are being monitored. Thus, a parent sleeping in another room generally may not monitor the glucose activity and generally will not be alerted when the child's glucose levels become dangerously low (hypoglycemic) or high (hyperglycemic).

Similarly, requiring a separate RMD for each sensor module may be undesirable. For example, in an institutional (e.g., hospital) setting, it is typically necessary that a caregiver monitor a different RMD associated with each patient wearing a sensor module. This may be both costly and inconvenient. Moreover, the limited transmission distance associated with typical wireless continuous analyte monitoring systems makes it difficult, if not impossible, for a caregiver to monitor the analyte concentration levels of a patient located at greater distances from the caregiver's station.

It would be desirable to have a wireless continuous monitoring system that assists in addressing one or more of the above disadvantages.

### SUMMARY OF THE INVENTION

According to one embodiment of the present invention, a system for monitoring a concentration of an analyte in a fluid or tissue sample is disclosed. The system comprises a sensor module adapted to be borne on a patient. The sensor module includes a power supply adapted to provide a transmission power, a first transceiver adapted to transmit analyte-concentration information, and a memory. The system further comprises a remote monitoring device adapted to wirelessly communicate with the sensor module. The remote monitoring device includes a second transceiver adapted to receive the analyte-concentration information transmitted by the sensor module and adapted to transmit a signal to the sensor module confirming that the analyte-concentration information was received. The information is stored in the memory until the signal is received by the sensor module.

According to a process of the present invention, a method of monitoring a concentration of an analyte in a fluid or tissue sample is disclosed. The method comprises the act of obtaining a measurement corresponding with the analyte concentration using a sensor module. The sensor module includes a memory and a power supply adapted to supply a transmission power. The method further comprises the act of transmitting the measurement to a remote monitoring device using the transmission power. The method further comprises the act of, upon receiving the measurement, transmitting a signal from the remote monitoring device to the sensor module. The method further comprises the act of, upon receiving the signal, removing the measurement from the memory.

According to another embodiment of the present invention, a system for monitoring an analyte concentration of one or more patients is disclosed. The system comprises a first sensor module adapted to be borne on a first patient. The first sensor module includes a first transceiver adapted to transmit analyte-concentration data associated with the first patient. The system further comprises a second sensor module adapted to be borne on a second patient. The second sensor module includes a second transceiver adapted to transmit analyte-concentration data associated with the second patient. The system further comprises a remote monitoring device including a third transceiver adapted to receive the analyte-concentration data wirelessly transmitted by one or more of the first and second sensor modules. The first and second transceivers are adapted to transmit data to one another and to the remote monitoring device.

According to another embodiment of the present invention, a system for monitoring an analyte concentration of one or more patients is disclosed. The system comprises a sensor module adapted to be borne on a patient. The sensor module includes a transceiver adapted to transmit analyte-concentration data associated with the patient. The system further comprises more than one remote monitoring device. Each of the remote monitoring devices includes a second transceiver adapted to receive the analyte-concentration data wirelessly transmitted by the sensor module.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1 is a perspective view of the components of a system according to one embodiment of the present invention.

FIG. 2 is a functional block diagram of the components according to one embodiment.

FIG. 3 is a flow diagram detailing a method according to one embodiment of the present invention.

FIG. 4 is a flow diagram detailing a method according to another embodiment of the present invention.

FIG. 5a is a perspective view of a system including multiple sensor modules and a single remote monitoring device, according to one embodiment of the present invention.

FIG. 5b is a perspective view of a system including a single sensor module and multiple remote monitoring devices, according to another embodiment of the present invention.

FIG. 5c is a perspective view of a system including multiple sensor modules and multiple remote monitoring devices, according to another embodiment of the present invention.

FIG. 5d is a perspective view of a system including multiple sensor modules and multiple remote monitoring devices, according to another embodiment of the present invention.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATED EMBODIMENTS

The present invention is directed to a wireless continuous analyte monitoring system. The system may be used to assist in determining an analyte concentration in a fluid or tissue sample. Some examples of the types of analytes that may be collected and analyzed include glucose, lipid profiles (e.g., cholesterol, triglycerides, LDL, and HDL), microalbumin, fructose, lactate, or bilirubin. The present invention is not limited, however, to these specific analytes, and it is contemplated that other analyte concentrations may be determined. The analytes may be in, for example, a whole blood sample, a blood serum sample, a blood plasma sample, and/or other body fluids like ISF (interstitial fluid). One non-limiting example of a use of the wireless continuous monitoring system of the present invention is to determine the glucose concentration in a user's blood, plasma, or ISF. Although glucose is the analyte used in many of the examples described herein, it is contemplated that any suitable analyte may be used in the embodiments of the present invention.

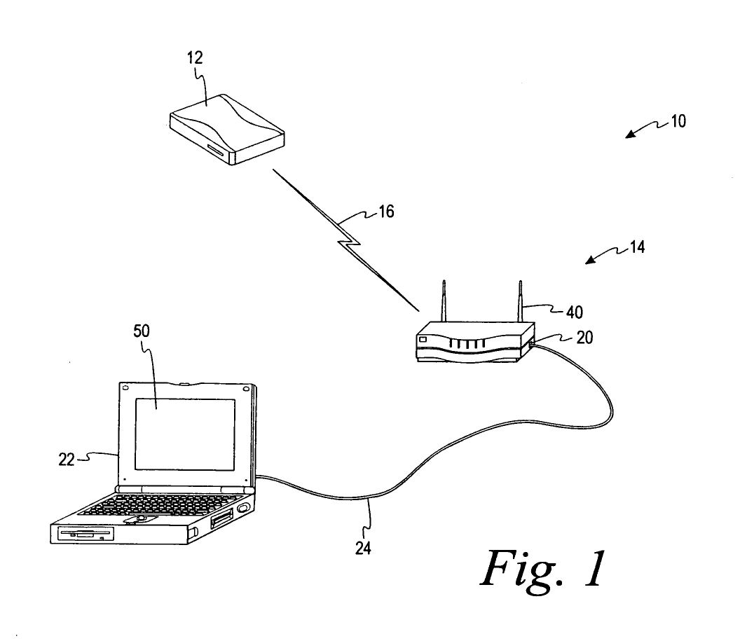

Referring to FIG. 1, a wireless continuous monitoring system 10 according to one embodiment of the present invention is shown. The system 10 includes a portable sensor module 12 and a remote monitoring device (RMD) 14. The sensor module 12 and the RMD 14 are linked by a wireless data communication link 16. The sensor module 12 may include any of a variety of devices capable of monitoring one or more selected physiological conditions or analyte (e.g., glucose) concentrations of a patient and to transmit data taken by the sensor module 12 to the RMD 14. The sensor module 12 may be borne on any suitable portion of the patient's body including, but not limited to, the patient's arm or abdomen. The sensor module 12 may be placed over the patient's skin (i.e., a transdermal sensor), or the sensor module or a portion thereof may be implanted under the patient's skin (i.e., an insertable sensor). The sensor module 12 may be removably secured to the patient's body using any suitable means including, but not limited to, adhesive, a bandage, a body or arm band, or the like.

The sensor module 12 may be maintained on a patient's body for extended periods of time. For example, the patient may wear the sensor module 12 for up to five days or more. To minimize the inconvenience to the patient, it is desirable for the sensor module 12 to be as small as possible. Small, battery-powered sensor modules allow the patient to move freely about while still permitting the continuous monitoring of relevant patient physiological conditions or analyte concentrations. Because of such size limitations and desired placement on a patient's body, it is often not feasible for the sensor module 12 to include a display for displaying data measurement values, a device capable of converting collected data into values useful to the patient, or the like. However, it is contemplated that a low power and/or an error indicator may be included on the sensor module 12. In one example, an LED is used to show that the sensor module 12 is working properly. The LED may display a green light if the sensor module 12 is functioning properly and a red light is the sensor module 12 is experiencing an error or problem. Other ways of indicating low power or errors in the sensor module 12 may also be used.

The RMD 14 may be carried by the patient in his or her pocket, worn around the patient's neck using a neck strap, shoulder strap, belt clip, or the like. Carrying the RMD 14 at all times, however, may be inconvenient, heavy, and/or bulky. Thus, the patient it may be desirable for the patient to remove the RMD 14 and place it in a stationary position (e.g., in the patient's home, workplace, or the like).

The RMD 14 may include a data port 20, which may be connected to a personal computer 22, a cellular telephone (not shown), or the like via a cable or cord 24. The data port 20 allows the RMD 14 to communicate with, for example, the personal computer 22 so that stored glucose readings, concentration trends, and corresponding information may be transferred to and displayed on the computer 22. The RMD 14 may also wirelessly communicate with the personal computer 22 (e.g., using Bluetooth or WiFi). The RMD 14 may also include a display (not shown) to display the measurement data, trends, and/or the like.

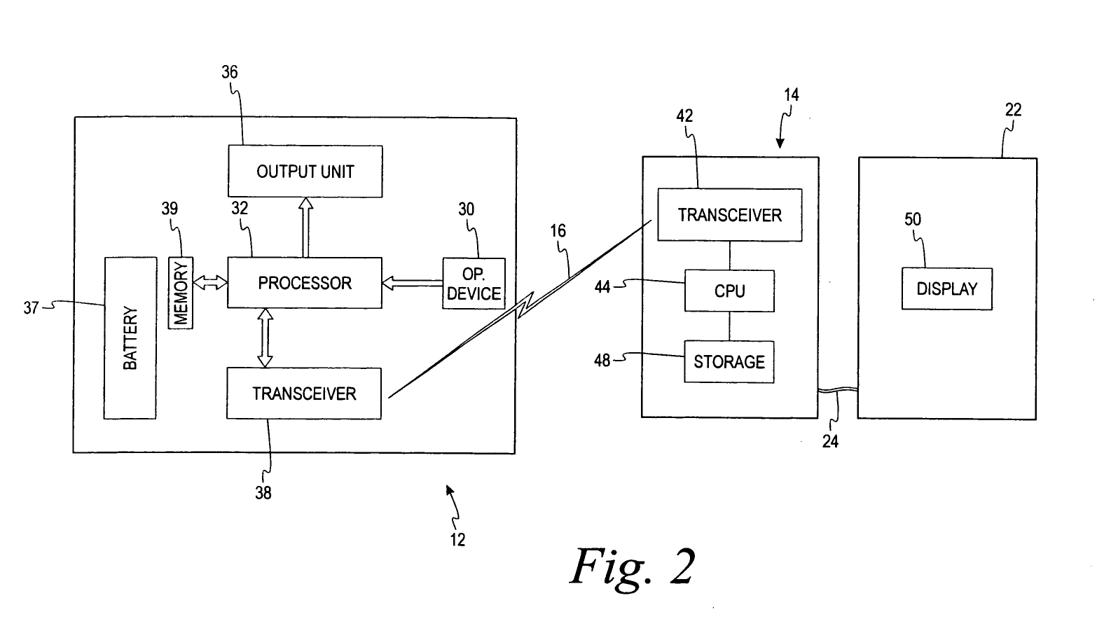

FIG. 2 is a block diagram of the functional components of the sensor module 12 and of the RMD 14. The sensor module 12 includes a unique identifier. The sensor module further includes an operation device 30 generally including elements required to operate the sensor module 12 and, thus, to measure a parameter correlating with, for example, the glucose concentration of the patient at the test site. The function of the operation device 30 is known to those skilled in the art and, therefore, will not be described further herein. The measurements are fed to a processor 32. The processor 32, which includes an analog-to-digital converter, converts the outputs received from the operation device 30 to a suitable digital form for transmission to the RMD 14. It is also contemplated that the measurements may be transmitted using direct analog transmission. Depending on the sensor module 12, the processor 32 may also include selected information regarding the patient, error checking means, or the like. The processor 32 may also process received data to a more useful form and/or have other suitable functions. The sensor module 12 further includes a memory unit 39, which may be separate from or associated with the processor 32. The sensor module 12 also includes a power-supply battery 37. The battery 37 may be rechargeable and is adapted to provide a transmission power to the RMD 14. A voltage monitor (not shown) may be integrated into the sensor module 12 to alert the patient in a timely manner of the need for battery exchange or recharge.

Although not necessary, the sensor module 12 may further include an output unit 36. The output unit 36 may issue an audio, visual (e.g., a light), and/or vibrational alarm if a certain condition(s) is met. For example, an alarm may be activated if a dangerous glucose value (i.e., dangerously high or low) is detected by the sensor module 12 Alternatively or additionally, the alarm may be activated if the sensor module 12 loses communication with the RMD 14. Thus, the output unit 36 may provide additional protection for the patient. The output unit 36 may be chosen for minimum size and consumption of battery 37.

The output from the processor 32 is provided to a transceiver 38. Any suitable type of transceiver 38 may be built into the sensor module 12 including, but not limited to the MaxStream® Xbee™ OEM RF module (MaxStream, Inc., Lindon, Utah) (“the MaxStream® module”), which operates generally within the ISM 2.4 GHz frequency band. Low-power systems, such as the MaxStream® module, may be desirable for use in longer-term analyte sensor modules.

The transceiver 38 provides the output from the processor 32 to a receiving antenna 40 (see FIG. 1), which also operates to receive wireless data. The transceiver 38 and/or the antenna 40 may be infra-red (IR) elements, radio frequency (RF) elements, or other suitable radiation transceiving elements. Radiation outputted by the transceiver 38 is, thus, received by one or more of the antenna 40 or other receiving devices suitably positioned on or in the RMD 14.

The receiving devices (e.g., antenna 40) are connected to a second transceiver 42 positioned within the RMD 14. Information received at the second transceiver 42 is provided to a CPU 44. The CPU 44 may perform further processing on the received information. The CPU 44 may selectively store such information, either in received form or in processed form, in temporary or bulk storage devices 48. Information is communicated between the transceiver 38 and the second transceiver 42 via the communication link 16, which is preferably a wireless communication link.

Furthermore, it may be desirable for a communications protocol facilitating communication with cellular phones, wireless computer networks, and the like, to be used. One non-limiting example of a communication protocol that may be used in the embodiments of the present invention is the 802.15.4 standard. The 802.15.4 standard offers a low data rate and power management functions to ensure low power consumption. Preferably, the transceiver 38 operates in the 2.4 GHz frequency band, in which there are 16 channels, each addressable by 16-bit or 64-bit addressing. For communication with cellular phones, the processor 32 and the CPU 44 are programmed to format data communicated across the communication link 16 according to any number of cellular data protocols, such as one compatible with the Short Message Service (SMS) of the CDMA or GSM cellular protocols. In one embodiment, data representative of the patient's monitored analyte concentrations may be sent as a text message across the SMS service to a cellular phone in lieu of or in addition to the RMD 14. Other information may also be sent, including information indicative of an alarm (for example, in response to a hypoglycemic or hyperglycemic glucose level), a signal indicative of a loss of communication with the RMD 14 (for example, if the patient leaves the RMD 14 at a location). Still other information that can be transmitted from the sensor module 12 to the RMD 14 is mentioned below and can also be transmitted to a cellular phone as a data message. In other aspects, the RMD 14 may communicate with a cellular phone by transmitting image data associated with the concentration levels (e.g., a graph showing historical measured levels over a period of time) or information indicating a loss of communication with the sensor module 12 (e.g., communication attempts between the RMD 14 and the sensor module 12 repeatedly failed over a period of time, whereupon the RMD 14 sends an SMS message to the cellular phone indicating the loss of communication with the sensor module 12).

The RMD 14 may then be linked to the computer 22 using the cable or cord 24. Upon linking the RMD 14 to the computer 22, the information is transferred from the storage device 48 to the computer 22 for further processing. Images indicative of such received information may be displayed on one or more displays (e.g., display 50 of FIG. 1) of the computer 22. Thus, the patient's relevant analyte concentration(s) and/or trends thereof may be monitored. The computer 22 may display the data in any suitable way including alpha-numerically, graphically, combinations thereof, or the like.

A unique sequence identifier (e.g., an index number) is assigned to each data measurement. As measurement data are collected, the sequence identifier is incremented. Thus, the software of the RMD 14 has a sequence identifier linked to the measurement data. The RMD 14 generally includes a real-time clock such that a time and/or date may be associated with each sequence identifier and, thus, with each data measurement. In another embodiment, the sensor module 12 includes a real-time clock. The time/date stamp may alternatively be used as the sequence identifier, as each time/date stamp will be unique. The sequence identifier is used to keep track of what data is transmitted from the sensor module 12 to the remote monitoring device 14. When measurement data are transmitted, the sequence identifier associated with the most recent data measurement not yet transmitted is logged by the sensor module 12. Alternatively, the sensor module 12 stores the range of sequence identifiers associated with the measurement data transmitted from the sensor module 12.

In another aspect of the present invention, the RMD 14 may transmit a signal to the sensor module 12 indicative of a request for data measured during a certain time period, such as the previous two hours. According to this aspect, the processor 32 of the sensor module 12 analyzes the time/date stamps (or the sequence identifier in embodiments where the time/date stamp is used as the sequence identifier) to determine which measurement data fall within the requested time period. The sensor module 12 then transmits to the RMD 14 the measurement data associated with the requested time period.

The transmission distance between the sensor module 12 and the remote monitoring device 14 may generally range from about 1 foot to about 100 feet. More specifically, the transmission distance may range from about 5 feet to about 30 feet. For example, the MaxSteam® module has a transmission distance of about 100 feet.

As discussed above, the RMD 14 is often not carried by the patient at all times. According to the present invention, if the sensor module 12 loses communication with the RMD 14, all measurement data collected during the period of lost communication are stored in the memory 39 of the sensor module 12 until communication is restored. Communication may be lost, for example, when the RMD 14 is out of range (e.g., when a patient fitted with the sensor module 12 is outside of the transmission distance from the RMD 14), when the RMD 14 is in an “off” position, when there is interference with the signal, when the patient is engaged in heavy exercise, or the like. Thus, when the sensor module 12 becomes sufficiently close to the RMD 14 such that the wireless exchange of data between the sensor module 12 and the RMD 14 is restored, the measurement data acquired during the period of non-communication are transferred from the memory 39 of the sensor module 12 into the second transceiver 42 of the RMD 14.

According to one embodiment, to further conserve power, a proximity sensor (e.g., a reed switch and a magnet) is utilized between the RMD 14 and the sensor module 12. When the sensor module 12 is within a certain distance from the RMD 14, communication between the sensor module 12 and the RMD 14 is restored. Alternatively or additionally, the proximity sensor may be used as a trigger to send data in an ultra low power version. In such an ultra low power version, to view the data, the display 50 must be within a certain proximity of the sensor module 12.

According to one aspect of the present invention, after the sensor module 12 transmits the measurement data to the RMD 14, the sensor module 12 awaits an acknowledgement signal from the RMD 14 that the measurement data have been successfully received. In one aspect, when the acknowledgement signal is received from the RMD 14, the sensor module 12 deletes the measurement data taken during the time period measured. In another aspect, the measurement data are not deleted until the next regular or scheduled transmission. The sensor module may store a range of sequence identifiers associated with the transmitted measurement data in the memory 39 or in the memory associated with the processor 32. The most recently stored sequence identifier is used as a “place holder” to keep track of what measurement data was already transmitted so that as the sensor module 12 continues to store subsequent measurement data, it has the ability to remember where the transmitted data ends and where the non-transmitted data begins. Thus, the sensor module 12 may continue to store subsequent measurement data while waiting for an acknowledgement from the RMD 14 that the transmitted data was received. Once that acknowledgment signal is received from the RMD 14 at the sensor module 12, the sensor module 12 may delete the measurement data associated with the stored sequence identifiers and all historical measurement data. Any newly stored but not yet transmitted measurement data are thus preserved in the sensor module 12 for transmission.

By buffering all measurement data and continuing to buffer measurement data following a transmission, loss of the measurement data is avoided in at least two ways. First, historical measurement data is preserved until it can be transmitted successfully to the RMD 14. In the event of a transmission failure, no measurement data is lost. Second, measurement data that is accumulated during or following a transmission is preserved until the next transmission can be made. The storage of the sequence identifiers prevents measurement data that is recorded during or following a transmission from being lost or erased. In other words, only that measurement data that is successfully transmitted and acknowledged by the RMD 14 as received will be deleted from the memory 39. All other measurement data (historical or future) is stored until it can be successfully transmitted.

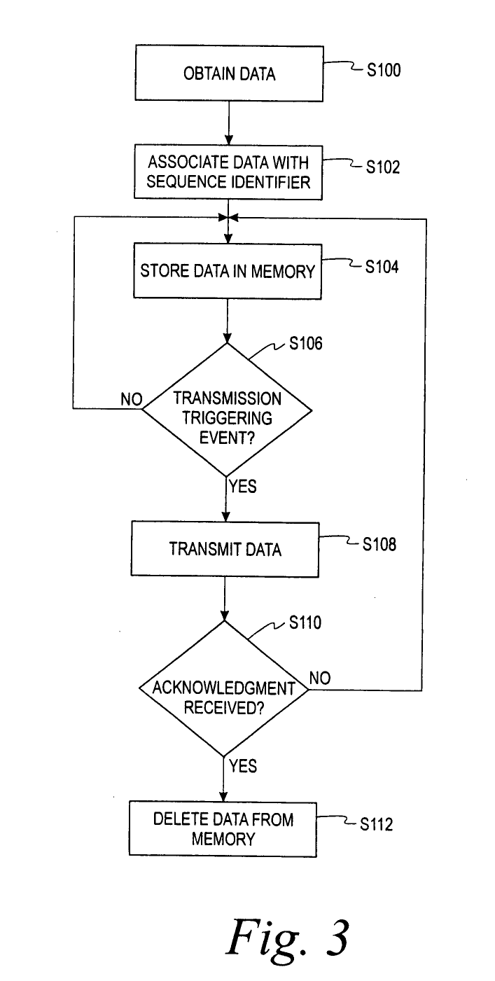

FIG. 3 illustrates a method according to one embodiment of the present invention. At step s100, the test sensor module 12 obtains measurement (e.g., analyte concentration) data. The measurement data is associated with a unique sequence identifier, as described above, at step s102. The measurement data and corresponding sequence identifier are stored in the memory 39 of the sensor module 12 at step s104. At determining step s106, whether a data-transmission triggering event has occurred is determined. A data-transmission triggering event may include occurrence of an alarm condition (e.g., exceeding a threshold indicating a hypoglycemic or hyperglycemic condition), passage of a predetermined time interval, an update request from the RMD 14, or the like. Thus, the sensor module 12, the RMD 14, or a combination thereof may trigger data transmission. If a data-transmission triggering event has not occurred, the measurement data continues to be stored in the memory 39 of the sensor module 12 (step s104). On the other hand, if a data-transmission triggering event has occurred, measurement data is transmitted from the sensor module 12 to the RMD 14 at step s108. Once the measurement data has been transmitted at step s108, whether the sensor module 12 has received an acknowledgement signal from the RMD 14 that the measurement data has been received is determined at step s110. If an acknowledgement signal has not been received, the measurement data is continued to be stored in the memory 39 of the sensor module 12 (step s104). If, on the other hand, an acknowledgement signal is received, the measurement data may be deleted from the memory 39 of the sensor module 12.

The storage capacities of the memory 39 of the sensor module 12 and the storage device 48 of the RMD. 14 match the particular applications for which they are intended. For example, the memory 39 of the sensor module 12 is used primarily for the intermediate storage of relatively small volumes of data, namely the measurement values stored since the last time that the sensor module 12 was in communication with the RMD 14. For example, the capacity of the memory 39 may be approximately 512 kilobits (kb). The memory 39 of the sensor module 12 may have the capacity to store measurement data for about 48 hours, depending on how often concentration measurements are taken. The capacity of the memory 39 may also be less than or greater than the examples provided. For example, the memory 39 of the sensor module 12 may be increased to store, for example, several days of measurement data. The memory 48 of the RMD 14 is generally substantially larger than the memory 39 of the sensor module 12 and may store data generated over long time intervals (at least one week).

The memory 39 or the memory associated with the processor 32 in the sensor module 12 may store other data (in addition to measurement data and the sequence identifier) indicative of the following types of modifiable information, alone or in any combination: (1) the time and/or date of the last successful transmission; (2) the time and/or date of the last attempted transmission; (3) one or more alarm thresholds corresponding to one or more analyte concentration measurements; (4) the type of alarm to be indicated by the output unit 36 (e.g., audible, visual, vibrational, or no alarm); (5) a desired power output level of the antenna 40; (6) the time interval during which the operation device 30 is to take a measurement (e.g., every hour or every two hours); (7) the type of analyte to be monitored; (8) the amount of power remaining in the battery 37; (9) the unique network address of the sensor module 12, which is used for transmitting information on an available channel in the communication link 16; or (10) relative aggregate data including, for example, data obtained during the most recent 1,000 minutes, 1,000 hours, 1,000 days, or other suitable time period. The sensor module 12 may use the amount of power remaining in the battery 37 to dynamically adjust the power output of the antenna 40. For example, if the battery 37 power level is low, the power output of the antenna 40 can be reduced to save battery power. The output unit 36 may indicate when the power level of the battery 37 becomes too low to sustain reliable functioning.

The memory 48 in the RMD 14 device may store data indicative of the following types of modifiable information, alone or in any combination: (1) the measurement data transmitted by the sensor module 12 and/or the corresponding sequence identifier(s); (2) one or more types of errors, such as corrupted data, partially received data, no data, and the like; (3) a command to send immediately or within a specified time period (a) all measurement data stored since the last transmission, (b) a selected range of measurement data, or (c) measurement data conforming to one or more criteria (such as (i) only those measurement data representing glucose concentrations exceeding a specified level, or (ii) the data measurement(s) corresponding to the highest measured analyte concentrations); (4) an analyte concentration threshold for triggering an alarm that is indicated by the output unit 36 of the sensor module 12; (5) one or more types of alarms to be indicated by the output unit 36 of the sensor module 12, such as an audible alarm, a visual alarm, a vibrational alarm, or no alarm; (6) a command to retransmit the measurement data; (7) a command for the sensor module 12 to erase part or all of its memory 39; (8) a command instructing the sensor module 12 to take a measurement immediately and transmit the measurement data to the RMD 14; (9) a command instructing the sensor module 12 to change its power output level and/or one or more power output levels (e.g., low, high, off); (10) data indicating that the RMD 14 will be unavailable for a specified or unspecified period of time and optionally the nature of the reason for such unavailability; (11) one or more time intervals during which the sensor module 12 is to take measurements (e.g., every two hours or every hour); (12) the type(s) of analyte(s) to be monitored by the sensor monitor 12 (e.g., glucose, lipids, fructose, bilirubin, and the like); (13) network administration information, such as (a) the network address of the RMD 14 or the sensor module 12 or (b) a command to the sensor module 12 to change its network address; (14) a command disallowing an attempted transmission by the sensor module 12 because it is an unauthorized device; (15) one or more SMS messages for transmission to a cellular telephone (such as a loss of communication with a sensor module, with an RMD), image data conveying information about the measurement data (e.g., a graph of measured analyte levels over a given period of time).

According to another embodiment of the present invention, the transmission power of the system 10 may be varied depending on the quality, level, and/or strength of communication between the sensor module 12 and the RMD 14. For example, the transmission power is decreased if the level of communication is strong or if battery power is low. Conversely, the transmission power is generally increased if the level of communication is weak. In the case where communication has been lost altogether and is not reestablished after a certain, predetermined time interval, the transmission power level may be reduced to its normal level, thereby assisting in conserving the life of the battery 37. In one embodiment, when communication has been lost altogether, the transmission power is reduced to a minimal level with an intermittent burst of high transmission power to seek to reestablish communication with the RMD 14. As described below, the transmission power can also be varied by the RMD 14 by issuing a command to the sensor module 12 to vary the power. In addition, the sensor module 12 may turn off the antenna 40 until the sensor module 12 needs to transmit data to the RMD 14.

In addition to the signals described above, the sensor module 12 may receive various other types of signals from the RMD 14. The signals sent from the RMD 14 to the sensor module 12 may include any one or more of the following: (1) a signal indicative of an acknowledgement that data transmitted from the sensor module 12 was received by the RMD 14; (2) a signal indicative of an error in the transmission of data received from the sensor module 12 (e.g., the data was corrupted during transmission); (3) a signal indicative of a command by the RMD 14 to the sensor module 12 to send now or within a specified time period (a) all measurement data stored since the last transmission, (b) a selected range of measurement data, or (c) measurement data conforming to one or more criteria (such as (i) only those measurement data representing glucose concentrations exceeding a specified level, or (ii) the data measurement(s) corresponding to the highest measured analyte concentrations); (4) a signal indicative of an analyte concentration threshold (e.g., a threshold indicating a hypoglycemic or hyperglycemic condition) for triggering an alarm that is indicated by the output unit 36 of the sensor module 12; (5) a signal indicative of the type of alarm to be indicated by the output unit 36 of the sensor module 12, such as an audible alarm, a visual alarm, a vibrational alarm, or no alarm; (6) a signal indicative of a command to the sensor module 12 to retransmit the measurement data; (7) a signal indicative of a command to erase part or all of the memory 39; (8) a signal indicative of a command instructing the sensor module 12 to take a measurement now and transmit the measurement data to the RMD 14; (9) a signal indicative of a command instructing the sensor module 12 to change its power output level; (10) a signal indicating that the RMD 14 will be unavailable for a specified or unspecified period of time and, optionally, the nature of the reason for such unavailability; (11) a signal indicative of the time interval during which the sensor module 12 is to take measurements (e.g., every two hours or every hour); (12) a signal indicative of the type(s) of analyte(s) to be monitored by the sensor monitor 12 (e.g., glucose, lipids, fructose, bilirubin, and the like); (13) a signal indicative of network administration, such as (a) a change in the network address of the RMD 14 or the sensor module 12 or (b) a command to the sensor module 12 to change its network address; (14) a signal disallowing the transmission because the sensor module 12 is an unauthorized device.

In response to receiving any of the above signals from the RMD 14, the sensor module may take the following respective actions: (1) erases the measurement data corresponding to the measurement data transmitted to and acknowledged as received by the RMD 14; (2) retransmits the measurement data; (3) transmits the requested measurement data; (4) changes the analyte concentration threshold for triggering an alarm, which threshold may optionally be stored in the memory 39; (5) changes the type of alarm to be indicated by the output unit 36, which type may optionally be stored in the memory 39; (6) retransmits the measurement data; (7) erases part or all of the memory 39; (8) takes a measurement and then transmits that measurement data to the RMD 14; (9) changes its power output level, which may optionally be stored in the memory 39 (e.g., the sensor module 12 enters a “power saving” mode); (10) continues to buffer measurement data in the memory 39 until the RMD 14 is available again and thereafter transmitting all buffered measurement data when the RMD 14 is expected to be available again; (11) changes the time interval during which the sensor module 12 is to take measurements, which time interval may optionally be stored in the memory 39; (12) monitors the requested type of analyte; (13) changes the network address associated with the sensor module 12; (14) attempts to transmit to another RMD in the network or to another sensor module.

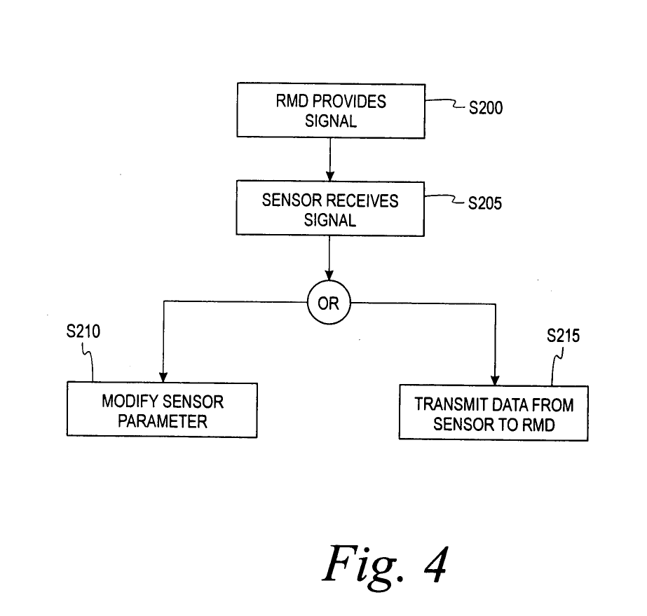

Referring to FIG. 4, a method according to one embodiment of the present invention is detailed. At step s200, the RMD 14 provides a signal to the sensor module 12. The signal may include any type of signal described above or combinations thereof. The sensor module 12 receives the signal at step s205. Depending on the type of signal produced and received, the sensor module 12 may modify a parameter of the sensor module software (step s210) or transmit measurement data from the sensor module 12 to the RMD 14 (step s215). Sensor parameters that may be modified at step s210 include, for example, analyte-concentration thresholds for triggering an alarm, the type of alarm, power output levels, the length of time between measurements, the type(s) of analyte(s) to be monitored, the sensor module's network address, combinations thereof, or the like. The transmitting data step (s215) may be conducted in accordance with the method illustrated in FIG. 3 and described above.

According to another embodiment, communication between the sensor module 12 and the RMD 14 are coordinated such that one transmits data at the same time the other is adapted to receive the data. This may be useful since power may be consumed by the devices when they are transmitting data as well as when they are receiving data. In one example, impromptu communication (e.g., an alarm condition) between the sensor module 12 and the RMD 14 may be preceded by a non-RF trigger to assist in synchronizing the communication between the sensor module 12 and the RMD 14.

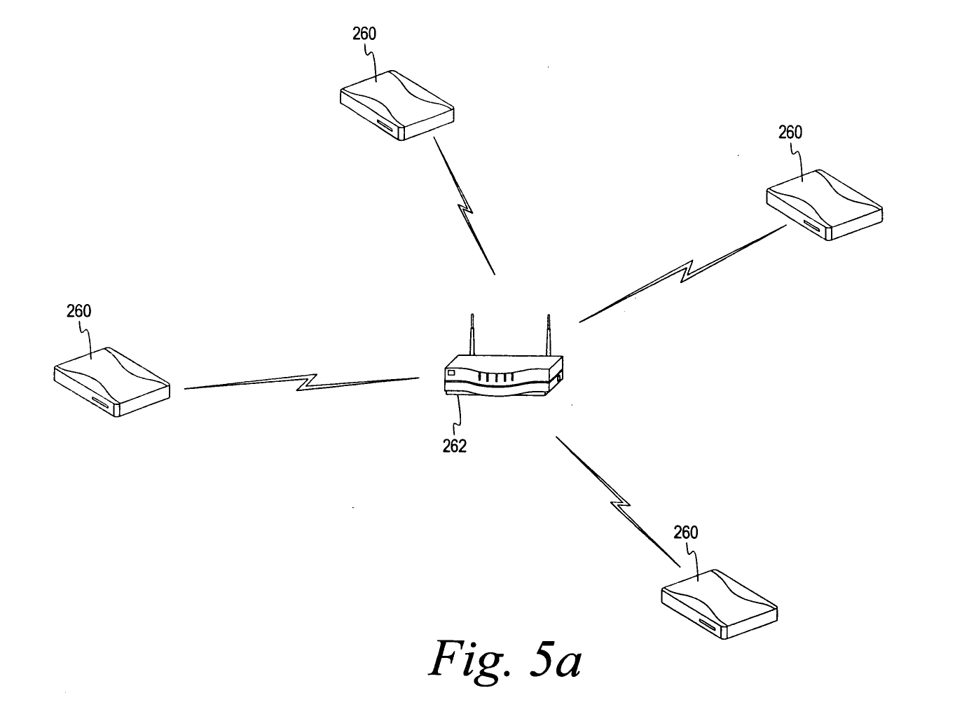

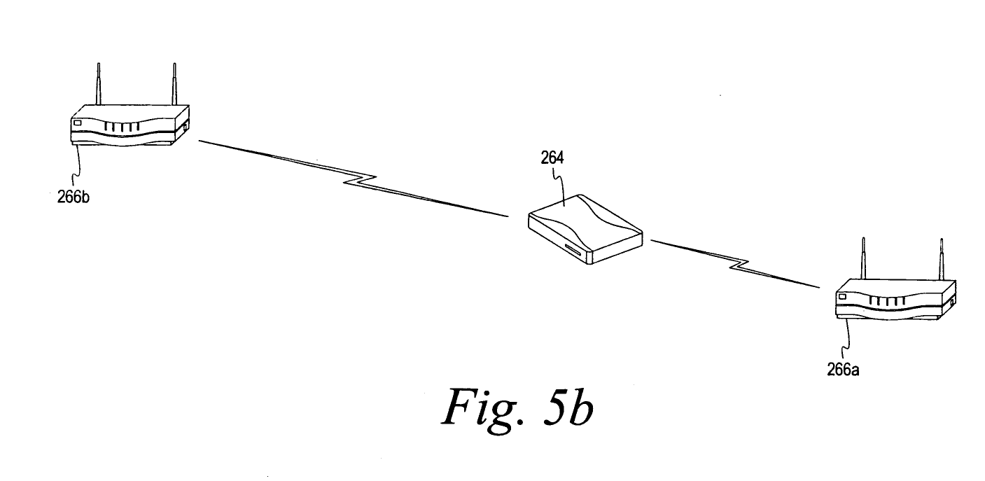

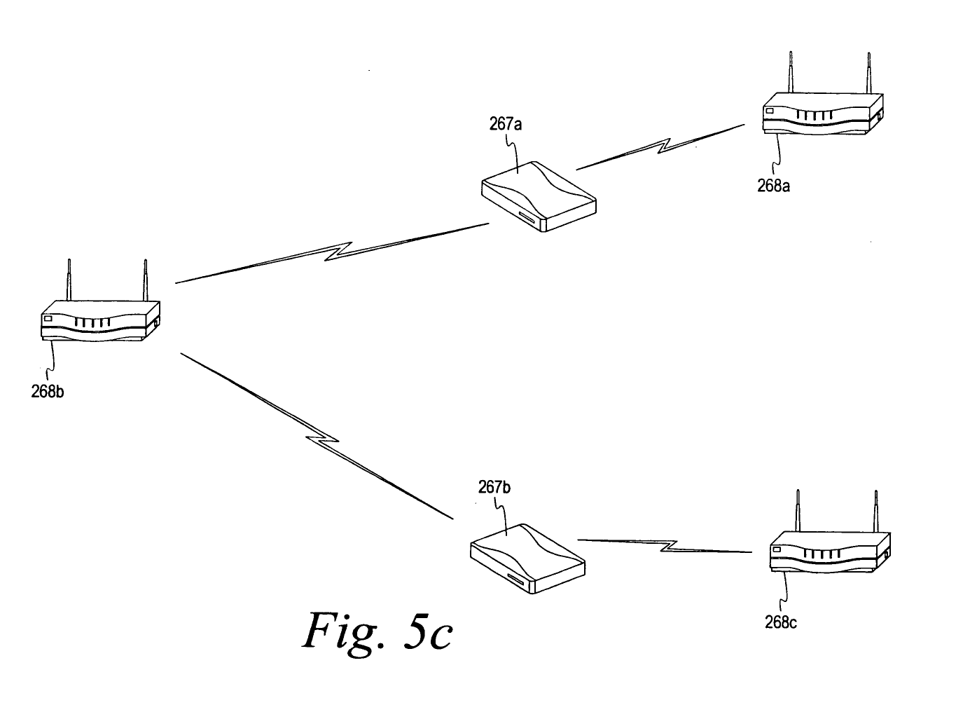

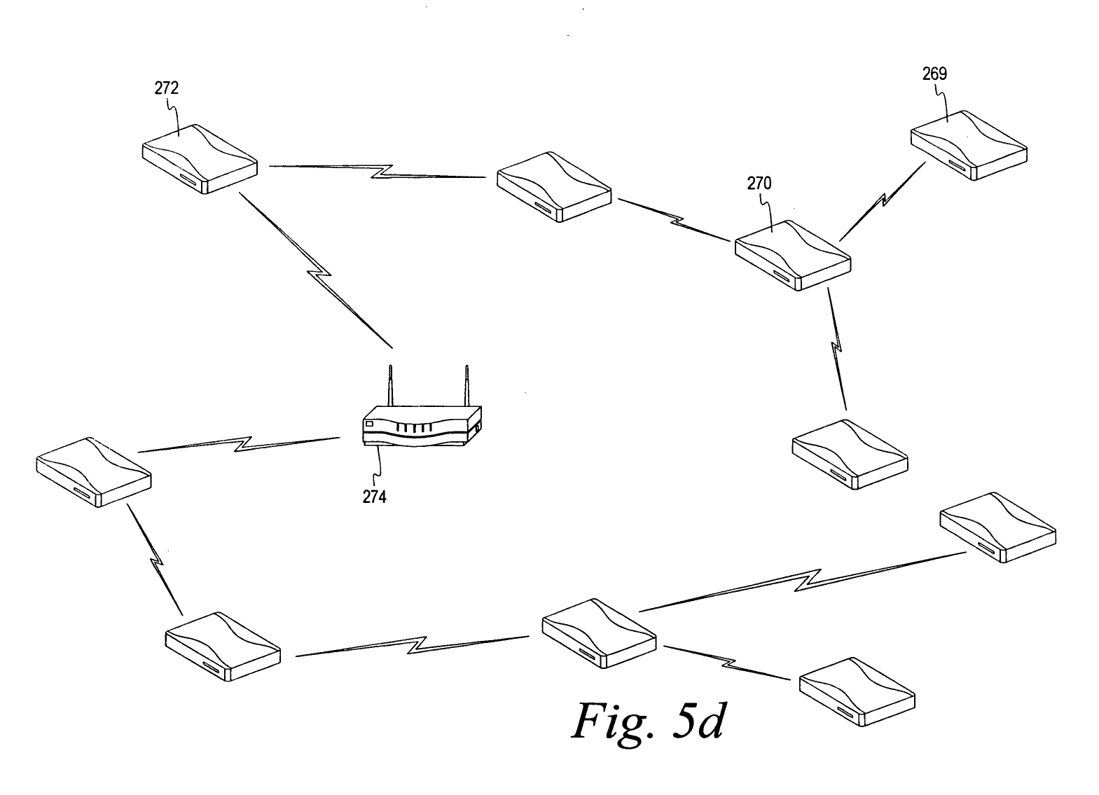

According to yet another aspect of the present invention, the wireless continuous monitoring systems of the present invention may be tailored to the needs of a user. For example, in FIGS. 5a-d, the systems of the embodiments of the present invention may include hardware capable of establishing point-to-point communication (see FIG. 5a), point-to-multipoint communication (see FIGS. 5b, 5c), peer-to-peer communication (see peer-to-peer link between sensor modules 269, 270 in FIG. 5d), or mesh communication (see FIG. 5d).

In FIG. 5a, multiple sensor modules 260 may transmit respective measurement data to a single RMD 262. This may be useful, for example, in an institutional (e.g., hospital) setting where a caregiver may monitor the analyte concentrations of several different patients on a single RMD 262.

In FIG. 5b, a single sensor module 264 may transmit measurement data to multiple RMDs 266a,b. The embodiment of FIG. 5b has multiple advantages. For example, if the sensor module 264 is borne by a child, a first RMD 266a may be located near the child such that measurement data from the sensor module 12 may be readily transmitted from the sensor module 12 to the first RMD 266a. An alarm on the first RMD 266a may issue if the child's glucose concentration is outside of the normal limits. A second RMD 266b located near the child's parent may also (or alternatively) issue an alarm, thereby alerting the parent that the child's glucose concentration is outside of the normal limits. This may be particularly useful, for example, when the child is sleeping, engaged in sports, playing outside, or the like. According to the embodiment of FIG. 5b, the child's parent need not be in the same room or even in hearing range of an alarm on the child's RMD 266a to know that the child's glucose concentration is at a dangerous level. Instead of or in addition to an alarm, the parent may monitor the child's glucose level in other suitable ways. For example, the second RMD 266b may include a display for displaying glucose levels, may be connected to a computer, or the like.

In embodiments where a wireless continuous monitoring system includes more than one RMD, as in FIG. 5b, it is contemplated that in certain circumstances, the sensor module 264 may be in communication with less than all of the RMDs 266a,b. In such a circumstance, the sensor module may include internal software allowing it to store measurement data in a memory (as described above) until it receives a signal from each of the RMDs 266a,b with which it is intended to communicate, indicating that all of the RMDs have successfully received the transmitted measurement data. Once all of the signals are received, the measurement data may be erased from the memory.

Because each sensor module has its own unique identifier associated therewith, multiple sensor modules may communicate with a single RMD. FIG. 5c shows multiple sensor modules 267a,b communicating with multiple receivers. In the embodiment of FIG. 5c, a first sensor module 267a is in communication with a first RMD 268a and a common RMD 268b. A second sensor module 267b is in communication with a second RMD 268c and the common RMD 268b. Such an embodiment may be desirable, for example, where two children in the same household are monitoring their glucose levels (e.g., on RMDs 268a, 268c) and a parent is monitoring the glucose levels of both children (e.g., on the central RMD 268b). Alternatively, this embodiment may be desirable in an institutional setting where two patients are individually monitoring their glucose levels (e.g., on RMDs 268a, 268c) and a caregiver is monitoring the glucose levels of both patients (e.g., on the central RMD 268b).

Referring now to FIG. 5d, a mesh system is shown in which data may be transmitted peer-to-peer between multiple sensor modules (e.g., sensor module 269 to sensor module 270) and from a sensor module (e.g., sensor module 272) to an RMD (e.g., RMD 274). The mesh system shown in FIG. 5d can reconfigure broken or blocked communication links by hopping from sensor module to sensor module until the destination is reached. Thus, a sensor module may transmit measurement data to an RMD outside its range by passing the measurement data through other sensor modules located at distances closer to the RMD via internal software. The transmission distance between the sensor module (e.g., sensor module 269) and the RMD 274 is thereby significantly increased. This embodiment may be particularly useful, for example, in institutional settings (e.g., hospitals) in which patients wearing sensor modules are located generally near each other but are located a substantial distance from the RMD 274. Thus, a single RMD 274 at, for example, a nurse's station, may allow the caregiver to monitor many sensor modules on many different patients.

In networks such as the mesh network shown in FIG. 5d enabling peer-to-peer communication, each sensor module can communicate uni-directionally or bi-directionally with other sensor modules in the network. In one aspect, if a memory in one sensor module becomes too full to store any further measurement data, that sensor module can “forward” subsequent measurement data to a proxy sensor module for storage until the sensor module has successfully transmitted its measurement data to an available RMD and erased its memory contents. The proxy sensor module can then retransmit the forwarded measurement data back to the sending sensor module when it has free memory space. In another aspect, if a sensor module loses communication with an RMD, it can pass its data to another proxy sensor module for transmission to an RMD. In this aspect, the originating sensor module also transmits identification information, such as its network address, along with the sequence identifiers and measurement data so that its origin can be ascertained by the RMD. The originating sensor module also sends a signal to the proxy sensor module indicative of a command to forward the forthcoming measurement data to the RMD. Thus, when the proxy sensor module receives the measurement data, it transmits the data to the RMD along with the identification information so that the RMD knows that this data is associated with a sensor module different from the sensor module sending the data.

Peer-to-peer configurations also allow the RMD to issue system-level commands or instructions to all sensor modules in the network without having to broadcast the command or instruction multiple times to each sensor module. In one aspect, the RMD sends a command or instruction to one sensor, which forwards it to all peers in the network in relay fashion.

In another embodiment, the RMD is adapted to receive data from a first sensor module to assist in calibrating a second sensor module. The first sensor module may, for example, have been used during a previous monitoring period, and the second sensor module may be used for a subsequent monitoring period. The RMD in this embodiment may inform a user when to replace the first sensor module with the second sensor module.

In yet another embodiment, the RMD may be adapted to communicate with an independent device that may be adjusted based on information transmitted from the RMD. For example, the RMD may communicate with a device connected to an intravenous drip providing, for example, glucose, insulin, drugs, pain killers, chemotherapeutic agents, or the like based on information received from the RMD.

The hardware of the sensor modules of the embodiments of the present invention may allow for multiple software-selectable frequencies. The 802.15.4 protocol operating in the 2.4 GHz frequency band allows for up to 16 different connections without sharing a channel. However, the 802.15.4 protocol includes two software addressing modes, 16-bit short and 64-bit IEEE addressing. By using software addressing within each channel, systems according to the present invention can accommodate 65,536 sensor modules (using 16-bit short addressing), each having its own network-addressable transceiver, on the same channel. In order to transmit on a channel, the sensor module would wait until it is addressed individually before using the channel. This may be useful, for example, in an institutional (e.g., hospital or hospital ward) setting where hundreds or even thousands of sensor modules could be operating at any given time.

In any of the foregoing aspects or embodiments described herein, data communicated across the wireless communication link between the sensor modules and the one or more RMD units may be encrypted for increased security and compliance with privacy regulations. The sensor module and RMD would include encryption and decryption modules that would also include appropriate keys for ciphering/deciphering the data. Alternately, the data communicated across the wireless communication link may be ciphered in such a way that renders it very difficult to recreate the original message without the proper cipher.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents, and alternatives falling within the spirit and scope of the invention.

### Alternative Embodiment A

A system for monitoring a concentration of an analyte in a fluid or tissue sample comprising:

a sensor module adapted to be borne on a patient, the sensor module including a power supply adapted to provide a transmission power, a first transceiver adapted to transmit analyte-concentration information, and a memory; and

a remote monitoring device adapted to wirelessly communicate with the sensor module, the remote monitoring device including a second transceiver adapted to receive the analyte-concentration information transmitted by the sensor module and adapted to transmit a signal to the sensor module confirming that the analyte-concentration information was received,
wherein the information is stored in the memory until the signal is received by the sensor module.

### Alternative Embodiment B

The system of Alternative Embodiment A, wherein the sensor module includes a transdermal test sensor or an insertable sensor.

### Alternative Embodiment C

The system of Alternative Embodiment A, wherein the analyte is glucose.

### Alternative Embodiment D

The system of Alternative Embodiment A, wherein the transmission power is fluctuated based on quality, level, or strength of the wireless communication.

### Alternative Embodiment E

The system of Alternative Embodiment A, wherein the remote monitoring device further comprises a device adapted to interpret the analyte-concentration information and an information storage device.

### Alternative Embodiment F

The system of Alternative Embodiment A, wherein the analyte-concentration information is stored in the memory when wireless communication with the remote monitoring device is lost and the analyte-concentration information is transmitted to the remote monitoring device when the wireless communication is restored.

### Alternative Embodiment G

The system of Alternative Embodiment A, wherein the analyte-concentration information includes measurement data having a sequence identifier being associated therewith, the sequence identifier being adapted to track whether the measurement data has been transmitted to the remote monitoring device.

### Alternative Embodiment H

The system of Alternative Embodiment A, wherein the analyte-concentration information is transmitted in response to a transmission triggering event.

### Alternative Embodiment I

The system of Alternative Process H, wherein the transmission triggering event includes exceeding a threshold analyte concentration, passage of a predetermined time interval, or a signal request from the remote monitoring device.

### Alternative Embodiment J

The system of Alternative Embodiment A, further comprising a second sensor module.

### Alternative Embodiment K

The system of Alternative Embodiment J, wherein the second sensor module is adapted to transmit analyte-concentration data to at least one of the sensor module and the remote monitoring device.

### Alternative Embodiment L

The system of Alternative Embodiment A, further comprising a second remote monitoring device adapted to wirelessly communicate with the sensor module, the second remote monitoring device including a third transceiver adapted to receive the analyte-concentration information transmitted by the sensor module.

### Alternative Embodiment M

The system of Alternative Embodiment A, wherein the remote monitoring device is adapted to be linked to a computer.

### Alternative Process N

A method of monitoring a concentration of an analyte in a fluid or tissue sample, the method comprising the acts of:

obtaining a measurement corresponding with the analyte concentration using a sensor module, the sensor module including a memory and a power supply adapted to supply a transmission power;

transmitting the measurement to a remote monitoring device using the transmission power;

upon receiving the measurement, transmitting a signal from the remote monitoring device to the sensor module; and

upon receiving the signal, removing the measurement from the memory.

### Alternative Process O

The method of Alternative Process N, further comprising varying the transmission power based on a level of communication between the sensor module and the remote monitoring device.

### Alternative Process P

The method of Alternative Process N, further comprising storing the measurement in the memory when communication with the remote monitoring device is lost, and wherein the act of transmitting the measurement to the remote monitoring device is performed when the communication is restored.

Alternative Process Q

The method of Alternative Process N, further comprising associating the measurement with a sequence identifier, the sequence identifier being adapted to track whether the measurement has been transmitted to the remote monitoring device.

### Alternative Process R

The method of Alternative Process N, wherein the act of transmitting the measurement to a remote monitoring device occurs in response to a transmission triggering event.

### Alternative Process S

The process of Alternative Process R, wherein the transmission triggering event includes exceeding a threshold analyte concentration, passage of a predetermined time interval, or a signal request from the remote monitoring device.

### Alternative Process T

The method of Alternative Process N, further comprising:

obtaining a second data measurement using a second sensor module, the second sensor module including a second memory; and

transmitting the second data measurement to one of the sensor module and the remote monitoring device.

### Alternative Process U

The method of Alternative Process T, wherein the sensor module is borne by a first patient and the second sensor module is borne by a second patient.

### Alternative Process V

The method of Alternative Process N, further comprising transmitting the data measurement to a second remote monitoring device.

### Alternative Process W

The method of Alternative Process N, further comprising linking the remote monitoring device to a computer.

### Alternative Embodiment X

A system for monitoring an analyte concentration of one or more patients, the system comprising:

a first sensor module adapted to be borne on a first patient, the first sensor module including a first transceiver adapted to transmit analyte-concentration data associated with the first patient;

a second sensor module adapted to be borne on a second patient, the second sensor module including a second transceiver adapted to transmit analyte-concentration data associated with the second patient; and

a remote monitoring device including a third transceiver adapted to receive the analyte-concentration data wirelessly transmitted by one or more of the first and second sensor modules,

wherein the first and second transceivers are adapted to transmit data to one another and to the remote monitoring device.

### Alternative Embodiment Y

A system for monitoring an analyte concentration of one or more patients, the system comprising:

a sensor module adapted to be borne on a patient, the sensor module including a transceiver adapted to transmit analyte-concentration data associated with the patient; and

more than one remote monitoring device, each of the remote monitoring devices including a second transceiver adapted to receive the analyte-concentration data wirelessly transmitted by the sensor module.

What is claimed is:

1. A system for monitoring a concentration of an analyte in a fluid or tissue sample comprising:
a sensor module adapted to be borne on a patient, the sensor module including a power supply adapted to provide a transmission power, a first transceiver adapted to transmit analyte-concentration information, and a memory; and
a remote monitoring device adapted to wirelessly communicate with the sensor module, the remote monitoring device including a second transceiver adapted to receive the analyte-concentration information transmitted by the sensor module and adapted to transmit a signal to the sensor module confirming that the analyte-concentration information was received,
wherein the information is stored in the memory until the signal is received by the sensor module.

2. The system of claim 1, wherein the sensor module includes a transdermal test sensor or an insertable sensor.

3. The system of claim 1, wherein the analyte is glucose.

4. The system of claim 1, wherein the transmission power is fluctuated based on quality, level, or strength of the wireless communication.

5. The system of claim 1, wherein the remote monitoring device further comprises a device adapted to interpret the analyte-concentration information and an information storage device.

6. The system of claim 1, wherein the analyte-concentration information is stored in the memory when wireless communication with the remote monitoring device is lost and the analyte-concentration information is transmitted to the remote monitoring device when the wireless communication is restored.

7. The system of claim 1, wherein the analyte-concentration information includes measurement data having a sequence identifier being associated therewith, the sequence identifier being adapted to track whether the measurement data has been transmitted to the remote monitoring device.

8. The system of claim 1, wherein the analyte-concentration information is transmitted in response to a transmission triggering event.

9. The system of claim 8, wherein the transmission triggering event includes exceeding a threshold analyte concentration, passage of a predetermined time interval, or a signal request from the remote monitoring device.

10. The system of claim 1, further comprising a second sensor module.

11. The system of claim 10, wherein the second sensor module is adapted to transmit analyte-concentration data to at least one of the sensor module and the remote monitoring device.

12. The system of claim 1, further comprising a second remote monitoring device adapted to wirelessly communicate with the sensor module, the second remote monitoring device including a third transceiver adapted to receive the analyte-concentration information transmitted by the sensor module.

13. The system of claim 1, wherein the remote monitoring device is adapted to be linked to a computer.

14. A method of monitoring a concentration of an analyte in a fluid or tissue sample, the method comprising the acts of:
obtaining a measurement corresponding with the analyte concentration using a sensor module, the sensor module including a memory and a power supply adapted to supply a transmission power;
transmitting the measurement to a remote monitoring device using the transmission power;
upon receiving the measurement, transmitting a signal from the remote monitoring device to the sensor module; and
upon receiving the signal, removing the measurement from the memory.

15. The method of claim 14, further comprising varying the transmission power based on a level of communication between the sensor module and the remote monitoring device.

16. The method of claim 14, further comprising storing the measurement in the memory when communication with the remote monitoring device is lost, and wherein the act of transmitting the measurement to the remote monitoring device is performed when the communication is restored.

17. The method of claim 14, further comprising associating the measurement with a sequence identifier, the sequence identifier being adapted to track whether the measurement has been transmitted to the remote monitoring device.

18. The method of claim 14, wherein the act of transmitting the measurement to a remote monitoring device occurs in response to a transmission triggering event.

19. The method of claim 18, wherein the transmission triggering event includes exceeding a threshold analyte concentration, passage of a predetermined time interval, or a signal request from the remote monitoring device.

20. The method of claim 14, further comprising:
obtaining a second data measurement using a second sensor module, the second sensor module including a second memory; and
transmitting the second data measurement to one of the sensor module and the remote monitoring device.

21. The method of claim 20, wherein the sensor module is borne by a first patient and the second sensor module is borne by a second patient.

22. The method of claim 14, further comprising transmitting the data measurement to a second remote monitoring device.

23. The method of claim 14, further comprising linking the remote monitoring device to a computer.

24. A system for monitoring an analyte concentration of one or more patients, the system comprising:
a first sensor module adapted to be borne on a first patient, the first sensor module including a first transceiver adapted to transmit analyte-concentration data associated with the first patient;
a second sensor module adapted to be borne on a second patient, the second sensor module including a second transceiver adapted to transmit analyte-concentration data associated with the second patient; and
a remote monitoring device including a third transceiver adapted to receive the analyte-concentration data wirelessly transmitted by one or more of the first and second sensor modules,
wherein the first and second transceivers are adapted to transmit data to one another and to the remote monitoring device.

25. A system for monitoring an analyte concentration of one or more patients, the system comprising:
a sensor module adapted to be borne on a patient, the sensor module including a transceiver adapted to transmit analyte-concentration data associated with the patient; and
more than one remote monitoring device, each of the remote monitoring devices including a second transceiver adapted to receive the analyte-concentration data wirelessly transmitted by the sensor module.
